(19) United States
(12) Patent Application Publication
(10) Pub. No.: US 2004/0241159 A1
(43) Pub. Date: Dec. 2, 2004

(54) Uses

(76) Inventor: Herve Maurice De Cellery D'Allens, Les Ulis (FR)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/468,492

(22) PCT Filed: Feb. 22, 2002
(86) PCT No.: PCT/GB02/00811

(30) Foreign Application Priority Data
Feb. 23, 2001 (GB) 0104554.1

(51) Int. Cl. A61K 39/395

(52) U.S. Cl. 424/143.1

(57) ABSTRACT

The present invention relates to the treatment of conditions associated with cephalic pain such as migraine, cluster headache, chronic paroxysmal hermicrania, headache associated with vascular disorders, headache associated with substances or their withdrawl, rebound headache and tension headache. In particular it relates to the use of a 5HT₁, receptor agonist in combination with an analgesic and an anti-emetic and/or gastroprokinetic agent.

The present invention relates to the treatment of conditions associated with cephalic pain such as migraine, cluster headache, chronic paroxysmal hemicrania, headache associated with vascular disorders, headache associated with substances or their withdrawal, rebound headache and tension headache. In particular it relates to the use of a 5HT₁ receptor agonist in combination with an analgesic and an anti-emetic and/or gastroprokinetic agent.

5HT₁ receptor agonists are well known in the art and the term is to be broadly understood to include 5HT₁ receptor agonists of all types including, but not limited to, 5HT₁-like receptor agonists, 5HT_{1B} receptor agonists, 5HT_{1D} receptor agonists and 5HT_{1F} receptor agonists. Particular reference is made to the compounds sumatriptan (described for example in GB Patent No. 2162522, incorporated herein by reference), naratriptan, rizatriptan, zolmitriptan, frovatriptan, eletriptan, almotriptan, avitriptan, donitriptan, alniditan, ALX-0646, LY334370, U1092291, IS159 and PNY142633.

Numerous clinical studies have demonstrated the effectiveness of sumatriptan in migraineurs. However, some migraineurs do not respond to treatment with sumatriptan alone.

In some forms of migraine, certain patients have found total or partial relief with the use of analgesics such as paracetemol and phenacetin. However, it has been reported (WO98/06392) that analgesics, when taken alone, are rarely effective in providing complete and rapid relief of all the symptoms of migraine, but that with a combination therapy of 5HT₁ receptor agonists and analgesics, their effectiveness is increased.

Oral administration constitutes the generally preferred route for administration of pharmaceuticals since this route is particularly convenient and acceptable to patients. Unfortunately, oral compositions may be associated with certain disadvantages in the treatment of conditions associated with cephalic pain. For example, such conditions, particularly migraine, are associated with nausea, vomiting and gastrointestinal dysfunction in the form of delayed gastric emptying.

Von Seggern et al. (headache, 1996, 493-502) describes the possiblity of “triple therapy” combining metoclopramide, an NSAID and an ergotamine preparation for the treatment of acute migraine. However, there is no suggestion that sumatriptan could be used in such a triple combination.

GB2325161 discloses the use of an anti-emetic and/or gastroprokinetic agent in combination with a 5HT_{1B/1D} receptor agonist for the treatment of the nausea and vomiting associated with migraine. There is no suggestion that such a combination improves the efficacy of the 5HT_{1B/1D} agonist as an anti-migraine treatment.

WO00/25778 discloses the combined use of metoclopramide and 5HT₁ agonists for the acute treatment of migraine and states that such a combination provides enhanced efficacy and less nausea but provides no data to substantiate such statements.

U.S. Pat. No. 6,077,539 describes the use of a specific dosage form comprising rapid availability metoclopramide and a long-acting NSAID for the treatment of migraine. The formulation is absent any 5HT₁ receptor agonist agent in order to avoid any “negative side effects linked to excessive vasoactivity in regions of the body not involved in the pathogenesis of migraine.”

Further, it has been shown in human volunteers that a combination of zolmitriptan, paracetamol and metoclopramide caused no clinically significant changes in the bioavailability or absorption of the three drugs.

Thus, it could be concluded that a triple combination of a 5HT₁ receptor agonist, analgesic and anti-emetic and/or gastroprokinetic would have no clinical advantage over the dual combination of a 5HT₁ receptor agonist plus an analgesic, the dual combination of a 5HT₁ receptor agonist plus an anti-emetic and/or gastroprokinetic agent, or the dual combination of an analgesic and an anti-emetic and/or gastroprokinetic agent.

However, a medicament to overcome the delayed gastric emptying as well as providing complete and rapid relief of all the symptoms of migraine would be advantageous.

It has now surprisingly been found that the use of a 5HT₁ receptor agonist in combination with an analgesic and an anti-emetic and/or gastroprokinetic agent is effective in the treatment of conditions associated with cephalic pain.

Thus, the present invention provides, in one aspect, the use of a 5HT₁ receptor agonist, or a pharmaceutically acceptable derivative thereof, with an analgesic or a pharmaceutically acceptable derivative thereof, and an anti-emetic and/or gastroprokinetic agent, or a pharmaceutically acceptable derivative thereof, in the manufacture of a medicament for oral administration for use in the treatment of conditions associated with cephalic pain.

By pharmaceutically acceptable derivative is meant any pharmaceutically acceptable salt, solvate, ester or amide, or salt or solvate of such ester or amide, of the 5HT₁ receptor agonist, the analgesic or the anti-emetic and/or gastroprokinetic agent, or any other compound which upon administration to the recipient is capable of providing (directly or indirectly) the 5HT₁ receptor agonist, the analgesic or the anti-emetic and/or gastroprokinetic agent or an active metabolite or residue thereof.

Suitable pharmaceutically acceptable salts according to the invention include acid addition salts formed with inorganic acids such as hydrochlorides, hydrobromides, phosphates and sulfates and with organic acids, for example tartrates, maleates, fumarates, succinates and sulfonates.

The combinations of the invention may also be useful as analgesics. They may be used to improve the condition of a host, typically of a human being, suffering from pain. They may be employed to alleviate pain In a host. Thus, the combinations of the invention may be used as a preemptive analgesic to treat acute pain such as muscculoskeletal pain, post operative pain and surgical pain, chronic pain such as chronic inflammatory pain (e.g. rheumatoid arthritis (RA) and osteoarthritis (OA), neuropathic pain (e.g. post herpetic neuralgia (PHN), trigeminal neuralgia, neuropathies associated with diabetes and sympathetically maintained pain) and pain associated with cancer and fibromyalgia. The combinations of the invention may also be used in the treatment or prevention of pain associated with Functional Bowel Disorders (e.g. Irritable Bowel Syndrome), non cardiac chest pain and non ulcer dyspepsia.

Suitable 5HT₁ receptor agonists for use according to the invention include sumatriptan, naratriptan, zolmitriptan, eletriptan, rizatriptan, frovatriptan, almotriptan, avitriptan, donitriptan, alniditan, ALX-0646, LY334370, U1092291, IS159 and PNY142633. Naratriptan and sumatriptan are preferred, with sumatriptan being particularly preferred. A preferred form of sumatriptan is the succinate salt, particularly the 1:1 succinate. The 5HT₁ receptor agonists may be used alone or in combination with each other.

Analgesics are well known in the art. Suitable analgesics for use according to the invention include adenosine A1 receptor agonists, opioids, para-aminophenol derivatives and non-steroidal anti-inflammatory drugs (NSAIDs). A1 receptor agonists have been described in the art and include compounds described in the published patent applications WO99/24449, WO99/24450, WO99/24451, WO97/43300, WO98/16539, WO98/04126, WO98/01459, EP0322242, GB2226027, EP222330, WO98/08855, WO94/0707 and WO99/67262. Opioids include alfentanil, buprenorphine, codeine, dextropropoxyphene, diamorphine, dihydrocodeine, fentanyl, methadone, morphine, oxycodone, levorphanol, pentazocine, pethidine, nefopam, flupirtin, meptazinol and tramadol. Para-aminophenol derivatives include paracetamol (also known as acetaminophen), propacetamol, phenacetin and acetanilide. NSAIDs include naproxen, ibuprofen, flurbiprofen, ketoprofen, dexketoprofen, fenoprofen, fenbufen, tolfenamic acid, mefenamic acid, tiaprofenic acid, indomethacin, oxaprozin, diclofenac, aceclofenac, sulindac, ketorolac, nabumetone, phenylbutazone, azapropazone, diflunisal, piroxicam, tenoxicam, salicylates such as aspirin, and COX-2 inhibitors such as celecoxib, rofecoxib, valdecoxib, parecoxib, JTE-522, etoricoxib (MK663), nimesulide, flosulide, COX189, etodolac, meloxicam, DFP, NS398, L-745337, 2-(4-ethoxy-phenyl)-3-(4-methanesulfonyl-phenyl)-pyrazolo[1,5-b]pyridazine, 8-acetyl-3-(4-fluorophenyl)-2-(4-methanesulfonylphenyl)-imidazo[1,2-a]pyridine, 4-[2-(3-fluorophenyl)-6-trifluoromethyl-pyrazolo[1,5-a]pyridin-3-yl]-benzenesulfonamide and N-isobutyl-4-[4-(methylsulfonyl)phenyl]-6-(trifluoromethyl)pyrimidin-2-amine. Particularly suitable analgesics for use according to the invention are aspirin, naproxen, ibuprofen, paracetamol, and their pharmaceutically acceptable salts or solvates. Aspirin is especially suitable. The analgesics may be used alone or in combination with each other.

Suitable anti-emetic and/or gastroprokinetic agents for use according to the invention include alizapride, alosetron, azasetron, batanopride, bemesetron, benzquinamide, bietanautine, bromopride, buclizine, chlorpromazine, cinitapride, cisapride, clebopride, cyclizine, dimenhydrinate, diphenidol, dolasetron, domperidone, dronabinol, fedotozine, fludorex, flumeridone, galdansetron, granisetron, itasetron, loxiglumide, lurosetron, meclizine, methallatal, metoclopramide, metopimazine, nabilone, naboctate, ondansetron, oxypendyl, palonsetron, pancopride, pipamazine, prochlorperazine, promethazine, scopolamine, sulpiride, thiethylperazine, thioproperazine, trimethobenzamide, tropisetron and zacoprid and their pharmaceutically acceptable salts or solvates. Particularly suitable anti-emetic and/or gastroprokinetic agents for use according to the invention include metoclopramide, cisapride, domperidone, and their pharmaceutically acceptable salts or solvates. Metoclopramide is especially suitable. The anti-emetic and/or gastroprokinetic agents may be used alone or in combination with each other.

It will be appreciated by those skilled in the art that certain of the compounds described herein may exist in stereoisomeric forms (i.e. they may contain one or more asymmetric carbon atoms or may exhibit cis-trans isomerism). The individual stereoisomers (enantiomers and diastereomers) and mixtures of these are included within the scope of the present invention.

Analgesics that exist in enantiomeric forms include (S)- and (R)-naproxen ((S)- and (R)-2-(6-methoxy-2-naphthyl)propionic acid), (S)- and (R)-ibuprofen ((S)- and (R)-2-(4-(2-methylpropyl-phenyl)propionic acid), (S)- and (R)-flurbiprofen ((S)- and (R)-2-(2-fluoro-biphenyl-4-yl-propionic acid), (S)- and (R)-ketoprofen ((S)- and (R)-2-(3-benzylphenyl)propionic acid), (S)- and (R)-fenoprofen ((S)- and (R)-2-(3-phenoxyphenyl)propionic acid), (S)- and (R)-tiaprofenic acid ((S)- and (R)-2-(5-benzyl-thien-2-yl)propionic acid), (S)- and (R)-sulindac ((S)- and (R)-(Z)-2-(5-fluor-2-methyl-1-(4-methylsulfinylbenzylidene)-inden-3-yl) acetic acid), (S)- and (R)-ketorolac ((S)- and (R)-5-benzoyl-2,3-dihydro-pyrrolizine-1-carboxylic acid), (S)- and (R)-azapropazone ((S)- and (R)-5-dimethylamino-9-methyl-2-propylpyrazolo-[1,2-a][1,2.4]benzotriazin-1,3(2H)-dione), and (S)- and (R)-etodolac ((S)- and (R)-1,8-diethyl-1,3,4,9-tetrahydropyrazolo[3,4-b]-indol-1-ylacetic acid).

Anti-emetic and/or gastroprokinetic agents that exist in enantiomeric forms include (S)- and (R)-alizapride ((S)- and (R)-6-methoxy-N-[[1-(2-propenyl)-2-pyrrolidinylmethyl-1H-benzotriazole-5-carboxamide), (S)- and (R)-azasetron ((S)- and (R)-6-chloro-3,4-dihydro-4-methyl-3-oxo-N-3-quinuclidinyl-2H-1,4-benzoxazine-8-carboxamide), (S)- and (R)-batanopride ((S)- and (R)-4-amino-5-chloro-N-[2-(diethylamino)ethyl]-2-[(1-methylacetonyl)oxy]benzamide), (S)- and (R)-benzquinamide ((S)- and (R)-2-(acetyloxy)-N,N-diethyl-1,3,4,6,7,11b-hexahydro-9,10-dimethoxy-2H-benzo[a]quinolizine-3-carboxamide, (S)- and (R)-buclizine ((S)- and (R)-1-[(4-chlorophenyl)phenyl-methyl]-4-[[4-(1,1-dimethylethyl)phenyl]methyl]-piperazine, (S)- and (R)-cinitapride ((S)- and (R)-amino-N-[1-(3-cyclohexen-1-ylmethyl)-4-piperidyl]-2-ethoxy-5-nitrobenzamide), and (S)- and (R)-cisapride ((S)- and (R)-cis-4-amino-5-chloro-N-{1-[3-(4-fluorophenoxy)propyl]-3-methoxy-4-piperidyl}-2-methoxybenzamide.

Compounds for use according to the invention may be administered simultaneously or sequentially and, when administration is sequential, the 5HT₁ receptor agonist, analgesic and anti-emetic and/or gastroprokinetic agent may be administered in any order.

Compounds for use according to the invention may be administered as the raw material but the active ingredients are preferably provided in the form of pharmaceutical formulations.

The active ingredients may be used either as separate formulations or as a single combined formulation. When combined in the same formulation it will be appreciated that the active ingredients must be stable and compatible with each other and the other components of the formulation. Therefore, pharmaceutical formulations comprising a combination as defined above together with a pharmaceutically acceptable diluent or carrier comprise a further aspect of the invention. When formulated separately they may be provided in any convenient formulation, conveniently in such manner as are known for such compounds in the art.

When used according to the present invention, the 5HT₁ receptor agonist is preferably other than zolmitriptan.

In a further embodiment of the invention, there is provided the use of a 5HT₁ receptor agonist, or a pharmaceutically acceptable derivative thereof, as hereinbefore described, wherein the 5HT₁ receptor agonist is sumatriptan. In a preferred embodiment, less than 100 mg of sumatriptan per unit dose is used.

In a particularly preferred embodiment, the analgesic is aspirin and the anti-emetic and/or gastroprokinetic agent is metoclopramide.

In a further aspect, the invention provides a method of treatment of a mammal, including a human, suffering from conditions associated with cephalic pain, comprising administering an effective amount of a pharmaceutical composition for oral administration comprising a 5HT₁ receptor agonist, such as sumatriptan or naratriptan, or a pharmaceutically acceptable derivative thereof, with an analgesic, such as aspirin, naproxen, ibuprofen, paracetamol, or a pharmaceutically acceptable derivative thereof, and an anti-emetic and/or gastroprokinetic agent, such as metoclopramide, or a pharmaceutically acceptable derivative thereof. The analgesic is preferably aspirin.

In a further embodiment of the invention, there is provided a method of treatment, as hereinbefore described, wherein the 5HT₁ receptor agonist is sumatriptan. In a preferred embodiment, less than 100 mg of sumatriptan per unit dose is used. In a particularly preferred embodiment, the analgesic is aspirin and the anti-emetic and/or gastroprokinetic agent is metoclopramide.

In a further embodiment of the invention, there is provided a method of treatment, as hereinbefore described, wherein the mammal is a human who has not responded to treatment using a 5HT₁ receptor agonist alone.

In a further embodiment of the invention, there is provided a method of treatment, as hereinbefore described, wherein the mammal is a human who has not responded to treatment using an NSAID alone.

In a further aspect, the invention provides a pharmaceutical composition for oral administration for use in the treatment of conditions associated with cephalic pain, comprising a 5HT₁ receptor agonist, such as sumatriptan or naratriptan, or a pharmaceutically acceptable derivative thereof, with an analgesic, such as aspirin, naproxen, ibuprofen, paracetamol, or a pharmaceutically acceptable derivative thereof, and an anti-emetic and/or gastroprokinetic agent, such as metoclopramide, or a pharmaceutically acceptable derivative thereof. The analgesic is preferably aspirin.

In a further embodiment of the invention, there is provided a pharmaceutical composition, as hereinbefore described, wherein the 5HT₁ receptor agonist is sumatriptan. In a preferred embodiment, less than 100 mg of sumatriptan per unit dose is used. In a particularly preferred embodiment, the analgesic is aspirin and the anti-emetic and/or gastroprokinetic agent is metoclopramide.

In a further aspect, the invention provides a pharmaceutical composition for oral administration for use in the treatment of conditions associated with cephalic pain comprising a 5HT₁ receptor agonist, such as sumatriptan or naratriptan, or a pharmaceutically acceptable derivative thereof, with an analgesic, such as aspirin, naproxen, ibuprofen, paracetamol, or a pharmaceutically acceptable derivative thereof, and an anti-emetic and/or gastroprokinetic agent, such as metoclopramide, or a pharmaceutically acceptable derivative thereof, characterised in that the effective dose of 5HT₁ receptor agonist is lower than when the 5HT₁ receptor agonist is administered alone. The analgesic is preferably aspirin.

In a further embodiment of the invention, there is provided a pharmaceutical composition, as hereinbefore described, wherein the 5HT₁ receptor agonist is sumatriptan. In a preferred embodiment, less than 100 mg of sumatriptan per unit dose is used. In a particularly preferred embodiment, the analgesic is aspirin and the anti-emetic and/or gastroprokinetic agent is metoclopramide.

In a further aspect, the invention provides a pharmaceutical composition for oral administration for use in the treatment of conditions associated with cephalic pain comprising a 5HT₁ receptor agonist, such as sumatriptan or naratriptan, or a pharmaceutically acceptable derivative thereof, with an analgesic, such as aspirin, naproxen, ibuprofen, paracetamol, or a pharmaceutically acceptable derivative thereof, and an anti-emetic and/or gastroprokinetic agent, such as metoclopramide, or a pharmaceutically acceptable derivative thereof, characterised in that the analgesic is used at a dose lower than that considered to produce an analgesic effect. The analgesic is preferably aspirin.

In a further embodiment of the invention, there is provided a pharmaceutical composition, as hereinbefore described, wherein the 5HT₁ receptor agonist is sumatriptan. In a preferred embodiment, less than 100 mg of sumatriptan per unit dose is used. In a particularly preferred embodiment, the analgesic is aspirin and the anti-emetic and/or gastroprokinetic agent is metoclopramide.

In a further aspect, the invention provides a pharmaceutical composition for oral administration for use in the treatment of conditions associated with cephalic pain comprising a 5HT₁ receptor agonist, such as sumatriptan or naratriptan, or a pharmaceutically acceptable derivative thereof, with an analgesic, such as aspirin, naproxen, ibuprofen, paracetamol, or a pharmaceutically acceptable derivative thereof, and an anti-emetic and/or gastroprokinetic agent, such as metoclopramide, or a pharmaceutically acceptable derivative thereof, characterised in that the effective dose of 5HT₁ receptor agonist is lower than when the 5HT₁ receptor agonist is administered alone and the analgesic is used at a dose lower than that considered to produce an analgesic effect. The analgesic is preferably aspirin.

In a further embodiment of the invention, there is provided a pharmaceutical composition, as hereinbefore described, wherein the 5HT₁ receptor agonist is sumatriptan. In a preferred embodiment, less than 100 mg of sumatriptan per unit dose is used. In a particularly preferred embodiment, the analgesic is aspirin and the anti-emetic and/or gastroprokinetic agent is metoclopramide.

It will be appreciated that reference to treatment is intended to include prophylaxis as well as the alleviation of established symptoms.

The 5HT₁ receptor agonist, analgesic and anti-emetic and/or gastroprokinetic agent may be co-administered in the form of separate pharmaceutical compositions for simultaneous and/or sequential use. Preferably, the 5HT₁ receptor agonist analgesic and anti-emetic and/or gastroprokinetic agent are administered as a single pharmaceutical composition for oral use comprising effective amounts of the active ingredients.

For oral administration, the pharmaceutical compositions may take the form of, for example, tablets or capsules prepared by conventional means with pharmaceutically acceptable excipients such as binding agents (e.g. pregelatinised maize starch, polyvinylpyrrolidone or hydroxypropyl methylcellulose); fillers (e.g. lactose, microcrystalline cellulose or calcium hydrogen phosphate); lubricants (e.g. magnesium stearate, talc or silica); disintegrants (e.g. potato starch or sodium starch glycollate); or wetting agents (e.g. sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, cellulose derivatives or hydrogenated edible fats); emulsifying agents (e.g. lecithin or acacia); non-aqueous vehicles (e.g. almond oil, oily esters, ethyl alcohol or fractionated vegetable oils); and preservatives (e.g. methyl or propyl-p-hydroxybenzoates or sorbic acid). The preparations may also contain buffer salts, flavouring, colouring and sweetening agents as appropriate.

Thus, the ratio of 5HT₁ receptor agonist to analgesic to anti-emetic and/or gastroprokinetic agent used in the uses, method or compositions according to the invention is in the range of 1:25:300 to 2:10000:5 to 400:10:1 (by weight), e.g. 1:20:12 to 10:2000:1 to 200:200:1, especially 5:9:1 or 10:9:1.

The amount of 5HT₁ receptor agonist used according to the instant invention is preferably in the range of 0.2 to 200 mg per unit dose. For example, when sumatriptan is employed, the amount of sumatriptan in the composition is preferably in the range of 5 to 100 mg, more preferably 25 or 50 mg expressed as the weight of free base.

The amount of analgesic used according to the instant invention is preferably in the range of 5 to 1000 mg per unit dose. For example, when aspirin is employed, the amount of aspirin in the composition is preferably in the range of 100 to 1000 mg, more preferably 300 to 900 mg, such as 600 or 900 mg.

The amount of anti-emetic and/or gastroprokinetic agent used according to the instant invention is preferably in the range of 0.5 to 60 mg per unit dose. For example, when metoclopramide is employed, the amount of metoclopramide in the composition is preferably in the range of 1 to 60 mg, more preferably 2 to 20 mg, such as 5 or 10 mg.

The unit dose (for example contained in one tablet according to the invention) may be administered up to, for example, 6 times a day depending upon the unit dose used, the nature and severity of the conditions being treated, and the age and weight of the patient.

In the following examples, the 5HT₁ receptor agonist may be replaced by any of the suitable 5HT₁ receptor agonists described herein. Thus, for example, sumatriptan may be replaced by naratriptan, zolmitriptan, eletriptan, rizatriptan, frovatriptan, almotriptan, avitriptan, donitriptan, alniditan, ALX-0646, LY334370, U1092291, IS159 or PNY142633.

Additionally, the analgesic may be replaced by any of the suitable analgesics described herein. Thus, for example, aspirin may be replaced by naproxen, ibuprofen or paracetamol.

Additionally, the anti-emetic and/or gastroprokinetic agent may be replaced by any of the suitable anti-emetic and/or gastroprokinetic agents described herein. Thus, for example, metoclopramide may be replaced by cisapride or domperidone.

The 5HT₁ receptor agonists, analgesic and anti-emetic and/or gastroprokinetic agents when used according to the instant invention may also be combined with other active agents.

The invention is further illustrated by the following non-limiting examples wherein the 5HT₁ receptor agonist is sumatriptan, the analgesic is aspirin and the anti-emetic and/or gastroprokinetic agent is metoclopramide.

### EXAMPLE 1

20 patients suffering from acute migraine were treated with a combination of 100 mg sumatriptan, 900 mg aspirin and 10 mg metoclopramide. 65% of patients were pain free within 2 hours of treatment.

### EXAMPLE 2

28 patients with a history of failed treatment with a 5HT₁ receptor agonist alone and/or an NSAID alone and suffering from acute migraine were treated with a combination of 50 mg sumatriptan, 900 mg aspirin and 10 mg metoclopramide. 79% of patients experienced headache relief within 2 hours of treatment.

The application of which this description and claims forms part may be used as a basis for priority in respect of any subsequent application. The claims of such subsequent application may be directed to any novel feature or combination of features described herein. This may take the form of product, composition, process or use claims and may include, by way of example and without limitation, one or more of the following claims:

1-17. (Cancelled).

18. A pharmaceutical composition for oral administration for the treatment of conditions associated with cephalic pain, comprising:
a 5HT₁ receptor agonist or a pharmaceutically acceptable derivative thereof;
an analgesic or pharmaceutically acceptable derivative thereof;
an anti-emetic or a pharmaceutically acceptable derivative thereof; and
optionally, a gastroprokenitic agent or a pharmaceutically acceptable derivative thereof.

19. The pharmaceutical composition of claim 18 wherein the effective dose of 5HT₁ receptor agoinst is lower than the dose when the 5HT₁ receptor agonist is administered alone.

20. The pharmaceutical composition of claim 18 wherein the effective dose of analgesic is lower than the dose when the analgesic is administered alone.

21. The pharmaceutical composition of claim 18 wherein the condition associated with cephalic pain is migraine in a patient who has not responded to treatment using a 5HT₁ receptor agonist alone.

22. The pharmaceutical composition of claim 18 wherein the condition associated with cephalic pain is migraine in a patient who has not responded to treatment using the analgesic alone wherein the analgesic is a NSAID.

23. The pharmaceutical composition of claim 18 wherein the 5HT₁ receptor agonist is other than zolmitriptan.

24. The pharmaceutical composition of claim 18 wherein the 5HT₁ receptor agonist is selected from the group consisting of sumatriptan or a pharmaceutically acceptable derivative thereof and naratriptan or a pharmaceutically acceptable derivative thereof.

25. The pharmaceutical composition of claim 24 wherein the 5HT₁ receptor agonist is sumatriptan or a pharmaceutically acceptable derivative thereof.

26. The pharmaceutical composition of claim 25 wherein less than 100 mg of sumatriptan per unit dose is used.

27. The pharmaceutical composition of claim 18 wherein the analgesic is selected from the group consisting of aspirin, naproxen, ibuprofen, pracetamol, and pharmaceutically acceptable derivatives thereof.

28. The pharmaceutical composition of claim 27 wherein the analgesic is aspirin or a pharmaceutically acceptable derivative thereof.

29. The pharmaceutical composition of claim 27 wherein the analgesic is naproxen or a pharmaceutical acceptable derivative thereof.

30. The pharmaceutical composition of claim 18 wherein the anti-emetic is metoclopramide or a pharmaceutically acceptable derivative thereof.

31. The pharmaceutical composition of claim 18 wherein the 5HT₁ receptor agonist is selected from the group consisting of sumatriptan, naratriptan, and pharmaceutically acceptable derivatives thereof; the analgesic is aspirin or a pharmaceutically acceptable derivative thereof; and the anti-emetic is metoclopramide or a pharmaceutically acceptable derivative thereof.

32. The pharmaceutical composition of claim 31 wherein the 5HT1 receptor agonist is sumatriptan or a pharmaceutically acceptable derivative thereof; the analgesic is aspirin or a pharmaceutically acceptable derivative thereof; and the anti-emetic is metoclopramide or a pharmaceutically acceptable derivative thereof.

33. A method of treatment of a mammal suffering from conditions associated with cephalic pain comprising administering an effective amount of a pharmaceutical composition of claim 18.

34. The method of treatment of claim 33 wherein the effective dose of 5HT₁ receptor agoinst is lower than the dose when the 5HT₁ receptor agonist is administered alone.

35. The method of treatment of claim 33 wherein the effective dose of analgesic is lower than the dose when the analgesic is administered alone.

36. The method of treatment of claim 33 wherein the condition associated with cephalic pain is migraine in a patient who has not responded to treatment using a 5HT₁ receptor agonist alone.

37. The method of treatment of claim 33 wherein the condition associated with cephalic pain is migraine in a patient who has not responded to treatment using the analgesic alone wherein the analgesic is a NSAID.

38. The method of treatment of claim 33 wherein the 5HT₁ receptor agonist is other than zolmitriptan.

39. The method of treatment of claim 33 wherein the 5HT₁ receptor agonist is selected from the group consisting of sumatriptan or a pharmaceutically acceptable derivative thereof and naratriptan or a pharmaceutically acceptable derivative thereof.

40. The method of treatment of claim 39 wherein the 5HT₁ receptor agonist is sumatriptan or a pharmaceutically acceptable derivative thereof.

41. The method of treatment of claim 40 wherein less than 100 mg of sumatriptan per unit dose is used.

42. The method of treatment of claim 33 wherein the analgesic is selected from the group consisting of aspirin, naproxen, ibuprofen, pracetamol, and pharmaceutically acceptable derivatives thereof.

43. The method of treatment of claim 42 wherein the analgesic is aspirin or a pharmaceutically acceptable derivative thereof.

44. The method of treatment of claim 42 wherein the analgesic is naproxen or a pharmaceutical acceptable derivative thereof.

45. The method of treatment of claim 33 wherein the anti-emetic is metoclopramide or a pharmaceutically acceptable derivative thereof.

46. The method of treatment of claim 33 wherein the 5HT₁ receptor agonist is selected from the group consisting of sumatriptan, naratriptan, and pharmaceutically acceptable derivatives thereof; the analgesic is aspirin or a pharmaceutically acceptable derivative thereof; and the anti-emetic is metoclopramide or a pharmaceutically acceptable derivative thereof.

47. The method of treatment of claim 46 wherein the 5HT₁ receptor agonist is sumatriptan or a pharmaceutically acceptable derivative thereof; the analgesic is aspirin or a pharmaceutically acceptable derivative thereof; and the anti-emetic is metoclopramide or a pharmaceutically acceptable derivative thereof.

48. The method of treatment of claim 33 wherein said mammal is a human.
